(19) United States
(12) Patent Application Publication
(10) Pub. No.: US 2003/0045846 A1
(43) Pub. Date: Mar. 6, 2003

(54) Pulp and superabsorbent composite for improved intake performance

(76) Inventors: Lawrence Howell Sawyer, Roswell, GA (US); Andrew Scott Burnes, Lawrenceville, GA (US); Connie Lynn Hetzler, Sparta, NJ (US)

Correspondence Address: KIMBERLY-CLARK WORLDWIDE, INC., 401 NORTH LAKE STREET, NEENAH, WI, 54956

(21) Appl. No.: 10/261,396

(22) Filed: Sep. 30, 2002

Related U.S. Application Data
(62) Division of application No. 09/192,221, filed on Nov. 16, 1998, now abandoned.
(60) Provisional application No. 60/068,534, filed on Dec. 23, 1997.

(51) Int. Cl. A61F 13/15

(52) U.S. Cl. 604/365; 604/374

(57) ABSTRACT

There is provided an expandable absorbent composite comprising pulp in an amount from about 28 to about 90 weight percent, superabsorbent material in an amount from about 8 two about 70 weight percent and a binder fiber in an amount from about 2 to about 20 weight percent, where the composite has a density of from about 0.1 g/cc to about 0.3 g/cc.

Such a composite may be used in personal care products like diapers, training pants, absorbent underpants, adult incontinence products, and the like.

This application claims priority from U.S. Provisional Application No. 60/068,534 filed Dec. 23, 1997.

### FIELD OF THE INVENTION

This invention relates to absorbent articles particularly absorbent structures which are useful in personal care products such as disposable sanitary napkins, diapers, or incontinence guards.

### BACKGROUND OF THE INVENTION

The desired performance objectives of personal care absorbent products include low or no leakage from the product, a dry feel to the wearer, and thinness as a means to provide comfort to the wearer. Current absorbent products, however, often fail to meet these objectives for a variety of reasons.

Leakage can occur, for example, due to insufficient uptake rate by layers intended to provide retention or distribution capability in the intake or target zone. Attempts to alleviate leakage occurring by this mechanism include absorbent articles that incorporate surge material structures located above (i.e., toward the wearer) the retention or distribution materials. U.S. Pat. No. 5,364,382 to Latimer discloses nonwoven materials such as meltblowns, bonded carded webs, and pulp coforms that receive and subsequently release liquid to the retention means. The material structures of Latimer utilize large denier resilient fibers blended with small denier wettable fibers to achieve rapid liquid uptake and rapid liquid release to the underlying retention storage material. Additionally, U.S. Pat. No. 5,490,846 to Ellis discloses layered structures to improve intake rates of surge materials.

Despite the development of surge materials that attempt to achieve rapid uptake and rapid release to the retention material, the objective of thinness remains to be satisfactorily reached. The cited surges are quite thick and when placed into the intake zone of the absorbent article can cause poor fit in the crotch region of the absorbent product upon initial wearing and can lead to several performance problems. Firstly, the product can leak due to gapping that is created by the bulky surge material. Secondly, the product is not comfortable to the wearer when a bulky material is utilized to provide the necessary void volume for uptake. There remains a need, therefore, for materials which will rapidly uptake an insult to the target area and either store the insult of release it for subsequent storage, and which also remain relatively thin prior to insult.

It is an object of this invention to provide a material for personal care products which rapidly uptakes an insult and which can store it or transfer it to an adjacent material for distribution or storage, and which remains relatively thin prior to such insult. It is another object of this invention to provide a personal care product which, prior to insult, is thin and comfortable for a wearer.

### SUMMARY OF THE INVENTION

The objectives of the invention are achieved by materials and products which have been designed to be very thin prior to insult and expand rapidly when insulted.

The invention is an expandable absorbent composite made from pulp in an amount from about 30 to about 90 weight percent, superabsorbent material in an amount from about 10 two about 70 weight percent and a binder in an effective amount, for example, from about 1 to about 20 weight percent, where the composite has a density of from about 0.1 g/cc to about 0.3 g/cc.

Such a composite may be used in personal care products like diapers, training pants, absorbent underpants, feminine hygiene products, adult incontinence products, and the like.

### BRIEF DESCRIPTION OF THE FIGURES

FIGS. 1, 2 and 3 are graphs of the void volume generation in various composites of this invention upon saturation.

### DEFINITIONS

“Hydrophilic” describes fibers or the surfaces of fibers which are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90° are designated “wettable” or hydrophilic, while fibers having contact angles equal to or greater than 90° are designated “nonwettable” or hydrophobic.

“Layer” when used in the singular can have the dual meaning of a single element or a plurality of elements.

“Liquid” means a nongaseous, nonparticulate substance and/or material that flows and can assume the interior shape of a container into which it is poured or placed.

As used herein the term “nonwoven fabric or web” means a web having a structure of individual fibers or threads which are interlaid, but not in an identifiable manner as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

As used herein the term “microfibers” means small diameter fibers having an average diameter not greater than about 75 microns, for example, having an average diameter of from about 0.5 microns to about 50 microns, or more particularly, microfibers may have an average diameter of from about 2 microns to about 40 microns. Another frequently used expression of fiber diameter is denier, which is defined as grams per 9000 meters of a fiber and may be calculated as fiber diameter in microns squared, multiplied by the density in grams/cc, multiplied by 0.00707. A lower denier indicates a finer fiber and a higher denier indicates a thicker or heavier fiber. For example, the diameter of a polypropylene fiber given as 15 microns may be converted to denier by squaring, multiplying the result by 0.89 g/cc and multiplying by 0.00707. Thus, a 15 micron polypropylene fiber has a denier of about 1.42 (15²×0.89×0.00707=1.415). Outside the United States the unit of measurement is more commonly the “tex”, which is defined as the grams per kilometer of fiber. Tex may be calculated as denier/9.

“Spunbonded fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 20 microns.

“Meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241. Meltblown fibers are microfibers which may be continuous or discontinuous, are generally smaller than 10 microns in average diameter, and are generally tacky when deposited onto a collecting surface.

As used herein, the term “coform” means a process in which at least one meltblown diehead is arranged near a chute through which other materials are added to the web while it is forming. Such other materials may be wood pulp, superabsorbent particles, cellulose or staple fibers, for example. Coform processes are shown in commonly assigned U.S. Pat. No. 4,818,464 to Lau and U.S. Pat. No. 4,100,324 to Anderson et al. Webs produced by the coform process are generally referred to as coform materials.

“Conjugate fibers” refers to fibers which have been formed from at least two polymer sources extruded from separate extruders but spun together to form one fiber. Conjugate fibers are also sometimes referred to as multicomponent or bicomponent fibers. The polymers are usually different from each other though conjugate fibers may be monocomponent fibers. The polymers are arranged in substantially constantly positioned distinct zones across the cross-section of the conjugate fibers and extend continuously along the length of the conjugate fibers. The configuration of such a conjugate fiber may be, for example, a sheath/core arrangement wherein one polymer is surrounded by another or may be a side by side arrangement, a pie arrangement or an “islands-in-the-sea” arrangement. Conjugate fibers are taught, for example, in U.S. Pat. No. 5,382,400 to Pike et al. and may be used to produce crimp in the fibers by using the differential rates of expansion and contraction of the two (or more) polymers. Such fibers may also be splittable. Crimped fibers may also be produced by mechanical means and by the process of German Patent DT 25 13 251 A1. For two component fibers, the polymers may be present in ratios of 75/25, 50/50, 25/75 or any other desired ratios. The fibers may also have shapes such as those described in U.S. Pat. No. 5,277,976 to Hogle et al. which describes fibers with unconventional shapes.

The methods for making conjugate fibers are well known and need not be described herein in detail. To form a conjugate fiber, generally, two polymers are extruded separately and fed to a polymer distribution system where the polymers are introduced into a segmented spinneret plate. The polymers follow separate paths to the fiber spinneret and are combined in a spinneret hole which comprises either two or more concentric circular holes thus providing a sheath/core type fiber or a circular spinneret hole divided along a diameter into two parts to provide a side-by-side type fiber. The combined polymer filament is then cooled, solidified and drawn, generally by a mechanical rolls system, to an intermediate filament diameter and collected. Subsequenty, the filament is “cold drawn”, at a temperature below its softening temperature, to the desired finished fiber diameter and is crimped/textured and cut into a desirable fiber length. Conjugate fibers can be cut into relatively short lengths, such as staple fibers which generally have lengths in the range of 25 to 51 millimeters (mm) and short-cut fibers which are even shorter and generally have lengths less than 18 millimeters. See, for example, U.S. Pat. No. 4,789,592 to Taniguchi et al. and U.S. Pat. No. 5,336,552 to Strack et al, both of which are incorporated herein by reference in their entirety.

“Bonded carded web” refers to webs that are made from staple fibers which are sent through a combing or carding unit, which breaks apart and aligns the staple fibers in the machine direction to form a generally machine direction-oriented fibrous nonwoven web. Such fibers are usually purchased in bales which are placed in a picker which separates the fibers prior to the carding unit. Once the web is formed, it then is bonded by one or more of several known bonding methods. One such bonding method is powder bonding, wherein a powdered adhesive is distributed through the web and then activated, usually by heating the web and adhesive with hot air. Another suitable bonding method is pattern bonding, wherein heated calender rolls or ultrasonic bonding equipment are used to bond the fibers together, usually in a localized bond pattern, though the web can be bonded across its entire surface if so desired. Another suitable and well-known bonding method, particularly when using conjugate staple fibers, is through-air bonding.

“Airlaying” is a well known process by which a fibrous nonwoven layer can be formed. In the airlaying process, bundles of small fibers having typical lengths ranging from about 2 to about 19 millimeters (mm) are separated and entrained in an air supply and then deposited onto a forming screen, usually with the assistance of a vacuum supply. The randomly deposited fibers then are bonded to one another using, for example, hot air or a spray adhesive.

“Personal care product” means diapers, training pants, absorbent underpants, feminine hygiene products and adult incontinence products.

### TEST METHODS

Material caliper (thickness): The caliper of materials, which is a measure of thickness, is measured at 0.05 psi with a Starret-type bulk tester, in units of centimeters.

Density: The density of the materials is calculated by dividing the weight per unit area of a sample in grams per square meter (gsm) by the caliper of the sample in millimeters (mm) at 0.05 psi (68.9 Pascals) and multiplying the result by 0.001 to convert the value to grams per cubic centimeter (g/cc). A total of three samples would be evaluated and averaged for the density values.

Absorption Time Index (ATI): In this test the absorbent capacity of a superabsorbent material is determined versus time for up to 200 minutes under light pressure, e.g. about 0.01 psi.

A one inch (25.4 mm) inside diameter cylinder with an integral 100 mesh stainless steel screen on one end is used to hold 0.16±0.005 grams of dry superabsorbent. The superabsorbent should be carefully placed in the cylinder so that superabsorbent does not stick to the sides of the cylinder. The cylinder should be tapped gently to more evenly distribute the superabsorbent on the screen. A 4.4 gram, 0.995 diameter plastic piston is then placed in the cylinder and the cylinder, piston and superabsorbent assembly weighed. The assembly is placed in a 3 inch by 3 inch (76.4 mm by 76.4 mm) fluid basin having a 0.875 weight percent NaCl saline solution to a depth of 1 cm. Tap the cylinder gently to remove any air trapped under it and maintain the saline solution depth at 1 cm throughout the test.

Use a timer capable of reading 200 minutes in one second intervals. Start the timer and after 5 minutes in the solution, remove the assembly and blot on absorbent paper. A preferred paper is Kleenex® Premium Dinner Napkins from Kimberly-Clark Corp. though any other effective paper may be used. In blotting, press the paper tightly against the cylinder to ensure good contact. Touch the cylinder three times to dry paper and there should be very little liquid removed the third time. Weigh the assembly and return assembly to the fluid basin. Blotting and weighing should take about 5 seconds and the timer should be kept running throughout the test. Take readings at 5, 10, 15, 30, 45, 60, 75, 90, 120, 160 and 200 minutes. Use fresh dry napkins for each reading.

After the final reading, calculate the grams of fluid absorbed per gram of superabsorbent. The amount of liquid absorbed at particular times divided by the amount absorbed at 200 minutes may be plotted versus time for a graphical representation of the absorption rate.

The ATI is calculated as follows:

ATI=(t₁₀+t₂₀+t₃₀+t₄₀+t₅₀+t₆₀+t₇₀+t₈₀+t₉₀)/9

where tₙ is the time in minutes at which n percentage of the absorbent capacity at 200 minutes is used, e.g. t₃₀ is the time at which 30 percent of the total capacity is used.

Gravimetric Absorbency Test (GAT): This test measures the time an absorbent composite takes to absorb an insult. The void volume and mass of the sample are also measured. Place a 6.8 cm diameter circle of material to be tested onto the sample platform of a GAT unit tester with the delivery point in the center of the sample. The GAT tester is available from M/K Systems, Inc., of Danvers, Mass. 01923 and is model no. M/K241 Serial no. G1048. After zeroing the instrument, set the pressure to 4 cm of positive hydrostatic pressure. Place a weight on the sample to provide 0.05 psi (68.9 Pascals) to the sample. Apply fluid to the sample, maintaining constant hydrostatic pressure and record the time it takes to intake 10 grams of 0.875 weight percent NaCl saline solution. Allow the sample to equilibrate for 30 minutes and repeat until the desired number of insults has been delivered. After the final insult, remove the sample and measure the mass and thickness while under 0.05 psi pressure.

### DETAILED DESCRIPTION OF THE INVENTION

The material structures of this invention have been designed to be very thin materials that expand rapidly when insulted. Therefore, when the user applies the absorbent article it is very thin and comfortable and facilitates good fit. As the material structure expands during use, it creates the necessary void volume to accommodate incoming fluid which in turn reduces the chances for leakage.

There are several embodiments of this invention described below. All have been designed to begin with a very thin structure which then expands upon contact with the insult fluid.

The material structure consists of modified pulp fiber, modified superabsorbent, and a small amount of binder, preferably thermally activated binder fiber. The material is initially produced and stabilized at a low density. The material is then compressed and fixed at a higher density as part of an absorbent system that meets the thinness requirements. When insulted, the compressed structure recovers a substantial portion of its as produced caliper, achieving an intermediate density between as produced and compressed states. The expandable composite provides the greatest benefit when used as part of an absorbent system that moves liquid out of the target zone.

Specific examples include structures containing pulp fiber, superabsorbent material, and thermoplastic binder fiber and were produced on a Dan Web airlay system. Composites within the scope of this invention contained the Weyerhauser Company's NHB-416 crosslinked pulp fiber at 75% or 55% by weight, and conjugate fibers by the Hoechst-Celanese Company called Celbond T105 at 5% by weight. Superabsorbent materials were Dow Chemical's AFA-130-53C, Stockhausen's W77553 or Stockhausen's FAV880A at 20% or 40% by weight. Materials were produced at low density (0.05 g/cc) and moderate density (0.1 g/cc) and thermally stabilized on line. Stockhausen's W77553 is a bulk polymerized polyacrylate with a hydrophobic surface treatment. Stockhausen's FAV880A is commercially available from the Stockhausen Company of Greensboro, N.C. 27406 and is a highly crosslinked surface superabsorbent. AFA 130-53C is a 850 to 1400 micron suspension polymerized polyacrylate particle from The Dow Chemical Company of Midland, Mich. Composites were compacted to the absorbent product target density of 0.20 g/cc by hot pressing in a shimmed carver press (80-100° C. for 1 minute). This pressing procedure resulted in materials that retained the 0.20 g/cc density over extended times. While other pressing procedures and other commercially available compressing procedures including continuous, on-line calendering, could also be utilized to produce the desired structures, lower temperature pressing (<50° C.) with and without moisture added, resulted in materials that rapidly rebulked under ambient conditions and would not maintain the desired thinness long enough to fall within the scope of this invention.

Higher temperature bonding (150° C.) resulted in samples that were over bonded and that did not rapidly expand when wetted and therefore did not result in the required void volume needed to fall within the scope of this invention. This contrasts with the sample that was bonded at better conditions (80-100° C.) and expands rapidly when wetted to greater than 80% of its uncompressed thickness and greater than 90% of its thickness when saturated if starting from the uncompressed state.

The preferred material structure, therefore, is a wet resilient but very hydrophilic, chemically cross-linked pulp fiber such as Weyerhaeuser NHB416 (28 to 90 weight percent), slow superabsorbent such as Dow AFA-130-53C or Stockhausen W77553 (8 to 70 weight percent), and a binder fiber such as sheath core conjugate fibers produced by Chisso, Hercules, Danaklon, or Hoechst-Celanese such as Celbond T105/T255 fibers.

By “slow rate” superabsorbent what is meant is a superabsorbent having an absorption time index (ATI) of at least 5 minutes and preferably more than 10 minutes. Note that though slow superabsorbents are preferred, fast or traditional superabsorbents will also function, as discussed below. Fast superabsorbents act to bind liquid rapidly, limiting spreading and wicking and reducing the overall distribution of liquid in the absorbent product but still providing very good intake performance.

The binder must be present in an effective amount to hold the structure together. While lower amounts may be possible a binder amount of between about 1 and about 20 weight percent are believed effective. Conjugate binder fibers are preferred though any binder known to be effective may be used. The binder must perform its function when the composite is compressed and maintain the composite in the higher density state until a liquid insult. Upon insult, the binder must allow the composite to expand to substantially its pre-compression caliper. If the binder is in fiber form it is preferable that the fibers be as fine as possible.

Moisture sensitive binders are available in powder, liquid, or fibrous form that may be activated using heat and/or small amounts of moisture. Binding systems may be polyvinyl alcohol adhesives, powders or fibers that dissolve in fluids. Some specific examples of polyvinyl alcohols have easily reversible crosslinks that allow changes in the adhesive property upon contact with the insult allowing the resilient structure to expand. Water sensitive hot melt adhesives could also be used that have time triggers based on controlled hydrophilicity or water triggerable polymers could be used such as base sensitive acrylics. Binders also include polyacrylic amides, polyacrylic acid and its copolymers, starch binders, cellulosic binders, and protein based binders.

Conjugate binder fibers which may be used in the practice of this invention may be crimped. One side of the conjugate fiber may have a water triggerable first component such as polyethylene oxide while the other side of the fiber may have a resilient fiber second component such as polyethylene terephthalate (PET). Many polymers are degradable in essentially plain water such as tap water which typically has a pH in the range of about 6.5 to about 8.5 and may serve as the water degradable portion of the conjugate fiber. Polymers can also be selected for the first component which are sensitive to or become degradable as a result of pH change, dissolved ion concentration change and/or temperature change in the aqueous environment.

Another mechanism which can be used to trigger water-degradability is ion sensitivity, where the term “ion” is given its conventional meaning of an atom or molecularly bonded group of atoms, which has gained or lost one or more electrons and consequently has a negative or positive electrical charge. Certain polymers contain acid-based (R—COO⁻) components which are held together by hydrogen bonding. In a dry state, these polymers remain solid. See for example, U.S. Pat. No. 4,419,403 to Varona which is incorporated herein by reference in its entirety.

Examples of polymers capable of degrading in aqueous mixtures or toilet water are poly (vinyl alcohol) graft copolymers supplied by the Nippon Synthetic Chemical Co., Ltd., Osaka, Japan, coded Ecomaty AX2000, AX10000 and AX-300G. The Nippon polymers are cold water soluble but somewhat slower in their rate of solubility than the Fuller polymers. Yet another first component polymer could be a polyether block amide, coded Pebax MX1074, supplied by Atochem (USA) located in Philadelphia, Pa. The Pebax MX1074 polymer is composed of epsilon-caprolactam (Nylon 12) and tetramethylene glycol monomers. These monomers are polymerized to make a series of polyether block amide copolymers. The Pebax polymer is not water soluble but is water-swellable, and therefore could also be used in a higher water volume environment as well. The Fuller polymers can be matched to a second component (core) polymer with a softening or melting temperature at least about 10° C. higher, such as would be the case with polypropylene. The Nippon or Atochem polymers can be matched with a higher melting temperature range second component polymer such as polypropylene or poly (butylene terephthalate).

More particularly, the expandable absorbent composite of this invention may have pulp in an amount from about 30 to about 80 weight percent, superabsorbent in an amount from about 10 to about 60 weight percent and binder in an amount from about 1 to about 10 weight percent. Still more particularly, the expandable absorbent composite may have pulp in an amount from about 55 to about 75 weight percent, superabsorbent in an amount from about 20 to about 40 weight percent and binder in an amount from about 3 to about 8 weight percent. Such composites must have a density after compression and before wetting of between about 0.1 g/cc and 0.30 g/cc, and preferably about 0.20 g/cc.

The attached Figures show the generation of void volume as a function of saturation for three material structures that fall within the scope of this invention. In the Figures, void volume is shown on the Y axis in units of cc/g and saturation is shown on the X axis in units of g/g.

### EXAMPLE 1

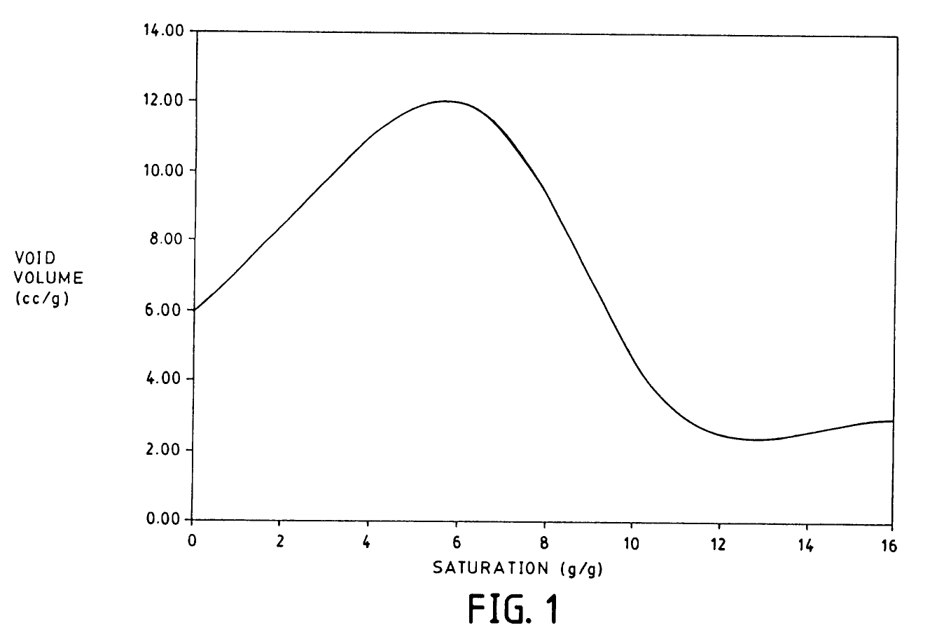

FIG. 1 is a graph of the void volume generation in a composite containing fast superabsorbent material Stockhausen FAV880 at 20 weight percent, chemically crosslinked pulp NHB416 from Weyerhaeuser at 75 weight percent, and Celbond T105 from Hoechst-Celanese at 5 weight percent upon saturation. The as-produced density was 0.04 g/cc and the sample consisted of 2 layers with a total plied basis weight of 400 gsm. The sample was pressed for 1 minute at 80 C. to a final density of 0.2 g/cc. The chart is a plot of void volume generation as a function of saturation. This chart illustrates that the sample begins with a void volume capability of 6 cc/g and doubles that capability to nearly 12 cc/g as the sample reaches 7 g/g saturation.

### EXAMPLE 2

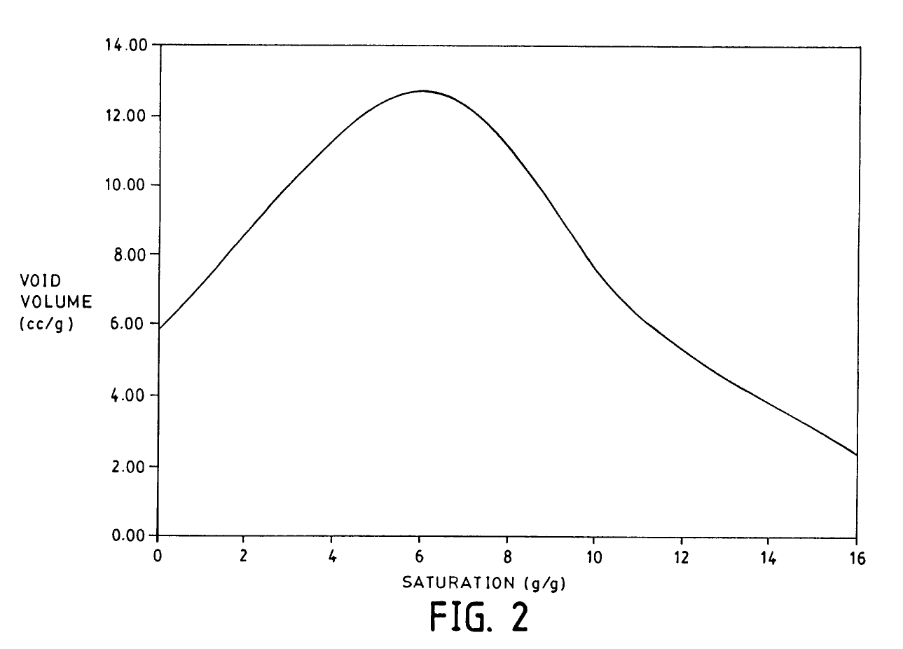

FIG. 2 shows comparable data for a layered composite that utilizes a slow Dow AFA-130-53C superabsorbent material in place of the Stockhausen superabsorbent FAV880 from FIG. 1 and the material was produced at a density of 0.055 g/cc. All other materials and pressing conditions are identical to those in Example 1. As can be seen in FIG. 2, this composition generates very similar void volume capability as a function of increased saturation.

### EXAMPLE 3

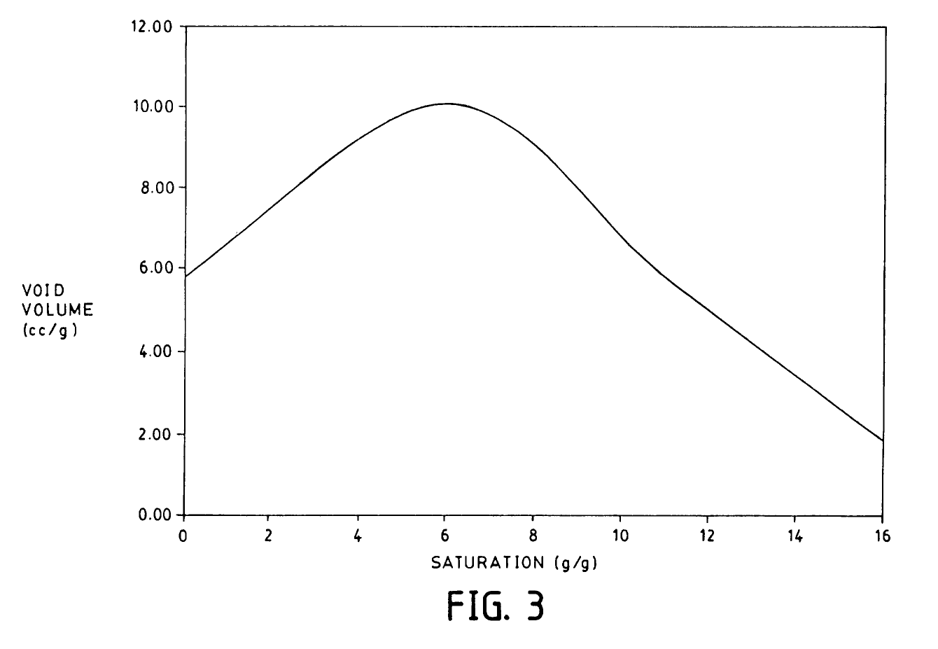

Another embodiment is a composite with the same composition and pressing conditions as in Example 2, but the composite had an as produced density of 0.11 g/cc. With this starting density, the void volume generation is only 10 cc/g at 7 g/g of saturation, but still falls within the scope of this invention. The void volume generation properties of this composite are illustrated in FIG. 3.

### EXAMPLE 4

This embodiment is a composite with the same materials and pressing conditions as Example 1 except that 55 weight percent pulp and 40 weight percent superabsorbent were used. This composite had an as produced density of 0.05 g/cc and a final density of 0.2 g/cc. There is no Figure representing the void volume generation of this Example.

### EXAMPLE 5

This embodiment is a composite with the same materials and pressing conditions as Example 2 except that 55 weight percent pulp and 40 weight percent superabsorbent were used. This composite had an as produced density of 0.053 g/cc and a final density of 0.2 g/cc. There is no Figure representing the void volume generation of this Example.

The Tables 1 and 2 show Gravemetric Absorbancy Test (GAT)data for the five Examples as well as for commercially available Huggies® diaper material. The Huggies® diaper material was taken from the retention material below the surge material in the frontal target zone of Huggies® for Him Ultratrim® diapers, size 3. These diapers are commercially available from the Kimberly-Clark Corporation, Dallas, Tex. These samples had a density of about 0.02 g/cc and contained either Stockhausen FAV880 or Dow 2035 commercially available superabsorbents in an amount of about 38 weight percent and Coosa 1654 pulp, available from Coosa Mills, Coosa, Ala., in an amount of about 62 weight percent.

Table 1 shows the Huggies® diaper results for two sets of samples as well as results for each of the Examples with two layers, as indicated. The Huggies® diaper samples had a basis weight of about 800 gsm and the Examples with two layers had a basis weight of about 400 gsm. The results in Table 2 show the Examples at a basis weight of about 800 gsm.

As can be seen from Table 1, all of the Example materials absorbed the 10 ml insults much more quickly than the Huggies® diaper on the first and second insults even though only half the mass of material was tested. The Example materials had about half the bulk (caliper) of the Huggies® diaper material. The Example materials used the available void volume more efficiently than the commercially available material as can be seen from the void volume data on the right hand side of Table 1. The Examples maintained high intake rates even after reaching 70 percent saturation.

Table 2 shows similar data as Table 1 but uses a basis weight and starting bulk for the Example materials which is about the same as the Huggies® diaper material. As can be seen from the Table 2 data, the Example material all absorbed all three 10 ml insults far faster than the Huggies® diaper material. Void volume utilization was also far improved.

The material of the Examples, while superficially somewhat similar to the Huggies® diaper material in composition, provided strikingly better performance. This is believed to be due to the generation of void volume which occurs as the Example materials expand upon liquid contact. This expansion is driven not only by normal superabsorbent expansion, as occurs in a Huggies® diaper, for example, but also by the expansion of the fibrous matrix in which the superabsorbent is contained. Superabsorbent gel blocking and subsequent liquid intake restriction is, therefore, eliminated or reduced in the composites of this invention. The composites of this invention, therefore, may be used as surge materials, as retention materials with a surge above as in conventional Huggies® diapers, or may be used as retention materials withoutsurge due to their superior absorbent properties.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims. In the claims, means plus function claims are intended to cover the structures described herein as performing the recited function and not only structural equivalents but also equivalent structures. Thus although a nail and a screw may not be structural equivalents in that a nail employs a cylindrical surface to secure wooden parts together, whereas a screw employs a helical surface, in the environment of fastening wooden parts, a nail and a screw may be equivalent structures.

TABLE 1
GRAVEMETRIC ABSORBENCY TEST - POINT SOURCE AT 4 CM POSITIVE HEAD

| | | | | | | | | | | | | |
|---|---|---|---|---|---|---|---|---|---|---|---|---|
| | BULK | | DEN- | | | | FINAL | FINAL | VOL- | VOID | FILLED | UNFILLED |
| | IN- | MASS | SITY | 1st inslt sec | 2nd inslt sec | 3rd inslt sec | MASS | BULK | UME | VOL | W | W |
| CODES | CHES | GRAMS | G/CC | SECONDS | SECONDS | SECONDS | GRAMS | INCHES | CC′S | CC′S | CC′S | CC′S |

HUGGIES

| | | | | | | | | | | | |
|---|---|---|---|---|---|---|---|---|---|---|---|
| 1 | 0.141 | 2.59 | 0.20 | 28.82 | 42.41 | 22.75 | 0.425 | 39.53 | 3.761 | 20.16 | 17.45 |
| 2 | 0.140 | 2.84 | 0.22 | 46.03 | 54.17 | 22.80 | 0.457 | 42.51 | 40.40 | 19.96 | 20.44 |
| 3 | 0.142 | 2.53 | 0.19 | 70.45 | 78.2 | 22.35 | 0.445 | 41.39 | 39.51 | 19.82 | 19.69 |
| AVG | 0.141 | 2.65 | 0.20 | 48.43 | 58.26 | 22.63 | 0.442 | 41.14 | 39.17 | 19.98 | 19.19 |

HUGGIES

| | | | | | | | | | | | | |
|---|---|---|---|---|---|---|---|---|---|---|---|---|
| 1 | 0.142 | 2.96 | 0.22 | 48.70 | 54.9 | 41.6 | 32.80 | 0.521 | 48.46 | 46.26 | 29.84 | 16.421 |
| 2 | 0.150 | 2.41 | 0.17 | 29.80 | 40.8 | 43.4 | 32.40 | 0.445 | 41.39 | 39.60 | 29.99 | 9.61 |
| 3 | 0.148 | 2.80 | 0.20 | 33.20 | 29.7 | 24.8 | 33.00 | 0.480 | 44.64 | 42.56 | 30.20 | 12.36 |
| AVG | 0.147 | 2.72 | 0.20 | 37.23 | 41.8 | 36.6 | 32.73 | 0.482 | 44.83 | 42.81 | 30.01 | 12.80 |

Ex 1, two layers

| | | | | | | | | | | | |
|---|---|---|---|---|---|---|---|---|---|---|---|
| 1 | 0.075 | 1.55 | 0.222 | 11.20 | 10.91 | 21.70 | 0.301 | 28.00 | 26.90 | 20.15 | 6.75 |
| 2 | 0.075 | 1.48 | 0.212 | 17.33 | 11.70 | 21.90 | 0.290 | 26.97 | 25.92 | 20.42 | 5.50 |
| 3 | 0.073 | 1.54 | 0.227 | 19.33 | 12.03 | 22.00 | 0.270 | 25.11 | 24.02 | 20.46 | 3.56 |
| AVG | 0.074 | 1.52 | 0.220 | 15.95 | 11.55 | 21.87 | 0.287 | 26.69 | 25.61 | 20.34 | 5.27 |

Ex 2, two layers

| | | | | | | | | | | | |
|---|---|---|---|---|---|---|---|---|---|---|---|
| 1 | 0.083 | 1.39 | 0.18 | 20.39 | 23.5 | 20.97 | 0.246 | 22.88 | 21.90 | 19.58 | 2.321 |
| 2 | 0.080 | 1.28 | 0.17 | 18.9 | 14 | 20.41 | 0.24 | 22.32 | 21.42 | 19.13 | 2.29 |
| 3 | 0.080 | 1.39 | 0.19 | 19.6 | 11.7 | 21.42 | 0.265 | 24.65 | 23.66 | 20.03 | 3.63 |
| AVG | 0.081 | 1.35 | 0.18 | 19.63 | 16.4 | 20.93 | 0.250 | 23.28 | 22.32 | 19.58 | 2.74 |

Ex 3, two layers

| | | | | | | | | | | | |
|---|---|---|---|---|---|---|---|---|---|---|---|
| 1 | 0.079 | 1.46 | 0.20 | 24.3 | 15.1 | 21.68 | 0.249 | 23.16 | 22.13 | 20.22 | 1.91 |
| 2 | 0.079 | 1.39 | 0.19 | 22.1 | 14 | 21.02 | 0.239 | 22.23 | 21.24 | 19.63 | 1.61 |
| 3 | 0.078 | 1.39 | 0.19 | 25 | 12.9 | 20.85 | 0.248 | 23.07 | 22.08 | 19.46 | 2.62 |
| AVG | 0.079 | 1.41 | 0.19 | 23.80 | 14 | 21.18 | 0.245 | 22.82 | 21.82 | 19.77 | 2.05 |

Ex 4, two layers

| | | | | | | | | | | | |
|---|---|---|---|---|---|---|---|---|---|---|---|
| 1 | 0.078 | 1.52 | 0.21 | 20.59 | 11.66 | 31.40 | 0.352 | 32.74 | 31.59 | 29.88 | 1.71 |
| 2 | 0.079 | 1.41 | 0.19 | 23.67 | 9.75 | 32.80 | 0.356 | 33.11 | 32.04 | 31.39 | 0.65 |
| 3 | 0.083 | 1.47 | 0.19 | 16.10 | 7.97 | 31.90 | 0.352 | 32.74 | 31.62 | 30.43 | 1.91 |
| AVG | 0.080 | 1.47 | 0.20 | 20.12 | 9.79 | 32.03 | 0.353 | 32.86 | 31.75 | 30.57 | 1.18 |

Ex. 4, two layers

| | | | | | | | | | | | | |
|---|---|---|---|---|---|---|---|---|---|---|---|---|
| 1 | 0.074 | 1.54 | 0.224 | 19.77 | 8.09 | 14.40 | 32.60 | 0.380 | 35.34 | 34.17 | 31.06 | 3.11 |
| 2 | 0.078 | 1.29 | 0.178 | 15.41 | 11.77 | 16.12 | 31.10 | 0.360 | 33.48 | 32.50 | 29.81 | 2.69 |
| 3 | 0.078 | 1.42 | 0.196 | 18.76 | 9.53 | 16.50 | 30.60 | 0.360 | 33.48 | 32.41 | 29.18 | 3.23 |
| AVG | 0.077 | 1.42 | 0.199 | 17.98 | 9.80 | | 31.43 | 0.367 | 34.10 | 33.03 | 30.02 | 3.01 |

Ex 5, two layers

| | | | | | | | | | | | |
|---|---|---|---|---|---|---|---|---|---|---|---|
| 1 | 0.075 | 1.41 | 0.202 | 18.74 | 7.68 | 21.96 | 0.295 | 27.44 | 26.37 | 20.55 | 5.82 |
| 2 | 0.074 | 1.39 | 0.202 | 21.07 | 8.91 | 21.50 | 0.293 | 27.25 | 26.20 | 20.11 | 6.09 |
| 3 | 0.079 | 1.24 | 0.169 | 14.74 | 58.4 | 20.80 | 0.240 | 22.32 | 21.38 | 19.58 | 1.82 |
| AVG | 0.076 | 1.35 | 0.191 | 18.18 | 7.48 | 21.42 | 0.276 | 25.67 | 24.65 | 20.07 | 4.88 |

TABLE 2
GRAVEMETRIC ABSORBENCY TEST - POINT SOURCE AT 4 CM POSITIVE HEAD

| | | | | | | | | | | | | |
|---|---|---|---|---|---|---|---|---|---|---|---|---|
| | BULK | | DEN- | | | | FINAL | FINAL | VOL- | VOID | FILLED | UNFILLED |
| | IN- | MASS | SITY | 1st inslt sec | 2nd inslt sec | 3rd inslt sec | MASS | BULK | UME | VOL | W | W |
| CODES | CHES | GRAMS | G/CC | SECONDS | SECONDS | SECONDS | GRAMS | INCHES | CC′S | CC′S | CC′S | CC′S |

Ex 1, four layer

| | | | | | | | | | | | | |
|---|---|---|---|---|---|---|---|---|---|---|---|---|
| 1 | 0.152 | 2.73 | 0.193 | 13.10 | 7.55 | 8.39 | 33.00 | 0.465 | 43.25 | 41.18 | 30.27 | 10.91 |
| 2 | 0.143 | 2.97 | 0.223 | 16.15 | 9.16 | 9.62 | 32.30 | 0.517 | 48.09 | 45.84 | 29.33 | 16.51 |
| 3 | 0.141 | 3.01 | 0.230 | 13.10 | 7.60 | 10.13 | 33.50 | 0.510 | 47.43 | 45.15 | 30.49 | 14.66 |
| AVG | 0.145 | 2.90 | 0.215 | 14.12 | 8.10 | | 32.93 | 0.497 | 46.26 | 44.06 | 30.03 | 14.03 |

Ex 2, four layers

| | | | | | | | | | | | | |
|---|---|---|---|---|---|---|---|---|---|---|---|---|
| 1 | 0.153 | 3.02 | 0.21 | 17.30 | 12.3 | 13.50 | 33.30 | 0.452 | 42.04 | 39.90 | 30.28 | 9.62 |
| 2 | 0.150 | 2.95 | 0.21 | 15.90 | 8.7 | 11.10 | 32.80 | 0.467 | 43.44 | 41.35 | 29.85 | 11.50 |
| 3 | 0.140 | 2.77 | 0.21 | 18.00 | 9.9 | 10.70 | 32.80 | 0.431 | 40.09 | 38.13 | 30.03 | 8.10 |
| AVG | 0.148 | 2.91 | 0.21 | 17.07 | 10.3 | 11.77 | 32.97 | 0.450 | 41.85 | 39.79 | 30.05 | 9.74 |

Ex 3, four layers

| | | | | | | | | | | | | |
|---|---|---|---|---|---|---|---|---|---|---|---|---|
| 1 | 0.139 | 2.97 | 0.23 | 19.90 | 6.6 | 12.00 | 33.30 | 0.450 | 41.85 | 39.75 | 30.33 | 9.42 |
| 2 | 0.135 | 2.97 | 0.24 | 19.50 | 9.8 | 12.20 | 33.40 | 0.452 | 42.04 | 39.94 | 30.43 | 9.51 |
| 3 | 0.153 | 2.98 | 0.21 | 19.60 | 8.8 | 12.50 | 33.50 | 0.450 | 41.85 | 39.74 | 30.52 | 9.22 |
| AVG | 0.142 | 2.97 | 0.22 | 19.67 | 8.4 | 12.23 | 33.40 | 0.451 | 41.92 | 39.81 | 30.43 | 9.38 |

Ex 4, four layers

| | | | | | | | | | | | | |
|---|---|---|---|---|---|---|---|---|---|---|---|---|
| 1 | 0.142 | 3.05 | 0.23 | 16.20 | 12.1 | 12.00 | 33.10 | 0.637 | 59.25 | 56.93 | 30.05 | 26.88 |
| 2 | 0.155 | 2.75 | 0.19 | 16.70 | 12.2 | 10.40 | 32.80 | 0.572 | 53.20 | 51.12 | 30.05 | 21.07 |
| 3 | 0.153 | 2.86 | 0.20 | 15.90 | 18.3 | 28.60 | 32.40 | 0.593 | 55.15 | 52.99 | 29.54 | 23.45 |
| AVG | 0.150 | 2.89 | 0.21 | 16.27 | 14.2 | 17.00 | 32.77 | 0.601 | 55.87 | 53.68 | 29.88 | 23.80 |

Ex 5, four layers

| | | | | | | | | | | | | |
|---|---|---|---|---|---|---|---|---|---|---|---|---|
| 1 | 0.152 | 2.65 | 0.187 | 16.42 | 8.49 | 9.63 | 32.60 | 0.560 | 52.09 | 50.08 | 29.95 | 20.13 |
| 2 | 0.154 | 2.77 | 0.193 | 16.05 | 10.43 | 10.42 | 32.00 | 0.520 | 48.37 | 46.26 | 29.23 | 17.03 |
| 3 | 0.158 | 2.77 | 0.189 | 14.27 | 13.06 | 17.40 | 32.50 | 0.515 | 47.90 | 45.80 | 29.73 | 16.07 |
| AVG | 0.155 | 2.73 | 0.190 | 15.58 | 10.66 | | 32.37 | 0.532 | 49.45 | 47.38 | 29.64 | 17.74 |

What is claimed is:

1. An expandable absorbent composite comprising pulp in an amount from about 30 to about 90 weight percent, superabsorbent material in an amount from about 10 two about 70 weight percent and a binder in an effective amount, wherein said composite has been compressed to a density of from about 0.1 g/cc to about 0.3 g/cc.

2. The composite claim 1 having pulp in an amount from about 40 to about 80 weight percent, superabsorbent in an amount from about 10 to about 50 weight percent and binder fiber in an amount from about 2 to about 10 weight percent.

3. The composite claim 1 having pulp in an amount from about 55 to about 75 weight percent, superabsorbent in an amount from about 20 to about 40 weight percent and binder fiber in an amount from about 3 to about 8 weight percent.

4. An expandable absorbent composite which has been compressed and which expands rapidly when wetted to greater than 80% of its uncompressed thickness and to greater than 90% of its uncompressed thickness when saturated.

5. A personal care product selected from the group consisting of diapers, training pants, feminine hygiene products, absorbent underpants and adult incontinence products comprising the material of claim 1.

6. The product of claim 5 wherein said personal care product is an adult incontinence product.

7. The product of claim 5 wherein said personal care product is a feminine hygiene product.

8. The product of claim 5 wherein said personal care product is a diaper.

9. A process of producing an expandable absorbent composite comprising the steps of:
blending together pulp in an amount from about 28 to about 90 weight percent, superabsorbent material in an amount from about 8 two about 70 weight percent and a binder fiber in an amount from about 2 to about 20 weight percent to form a web;
compressing said web at a temperature and time sufficient to produce a stabilized composite having a density between about 0.1 g/cc and about 0.3 g/cc.

10. The process of claim 9 wherein said web is heated at a temperature between about 80 and 100° C.
